(19) United States
(12) Patent Application Publication
(10) Pub. No.: US 2006/0177827 A1
(43) Pub. Date: Aug. 10, 2006

(54) Method computer program with program code elements and computer program product for analysing s regulatory genetic network of a cell

(76) Inventors: Mathaus Dejori, Munich (DE); Martin Stetter, Munich (DE)

Correspondence Address: STAAS & HALSEY LLP, SUITE 700, 1201 NEW YORK AVENUE, N.W., WASHINGTON, DC, 20005, US

(21) Appl. No.: 10/563,223

(22) PCT Filed: Jun. 28, 2004
(86) PCT No.: PCT/EP04/51266

§ 371 Date: Jan. 4, 2006

(30) Foreign Application Priority Data
Jul. 4, 2003 (DE) 103 30 280.8

(51) Int. Cl. C12Q 1/68; G06N 3/12; G06F 19/00

(52) U.S. Cl. 435/6; 702/20; 706/13

(57) ABSTRACT

A regulator genetic network of a cell is analyzed using a causal network after predefining a gene expression rate for a selected gene of the regulatory genetic network. The causal network is used to generate a resultant gene expression pattern relating to the genetic network for the predefined gene expression rate. The generated resultant gene expression pattern is subsequently compared with a predefined gene expression pattern of the regulatory genetic network.

### CROSS REFERENCE TO RELATED APPLICATIONS

This application is based on and hereby claims priority to German Patent Application No. 10330280.8 filed on Jul. 4, 2003, the contents of which are hereby incorporated by reference.

### BACKGROUND OF THE INVENTION

1. Field of the Invention

The invention relates to an analysis of a regulatory genetic network of a cell using a statistical method.

2. Description of the Related Art

Fundamentals of a regulatory genetic network of a cell are known from Stetter et al., Large-Scale Computational Modeling of Generic Regulatory Networks, Kluwer Academic Publisher, Netherlands, 2003. Such a regulatory genetic network should be taken in this document to mean in particular regulatory interactions between genes of a cell.

A genome, i.e. the human genetic substance, is estimated to comprise 20,000 to 40,000 genes, of which a biologically specified number in each case—depending on a specialization of a cell—are present in the cell in the form of a DNA or a part of a DNA.

A not necessarily contiguous section of this DNA containing the genetic code for a protein or also for a group of proteins or for creating a protein or a group of proteins is designated as a gene here. Overall the genes contain a genetic code for around a million proteins.

An interplay or the interactions between the genes as well as with the proteins represents the most important part of a machinery (regulatory genetic network) which underlies the development of a human body from a fertilized egg cell as well as all bodily functions.

It is also known from Stetter that so-called gene expression rates which form a gene expression pattern supply a description or representation of a regulatory genetic network or of a current status of the regulatory genetic network.

In simple terms or expressed more clearly the gene expression pattern of a cell thus represents a state of the regulatory genetic network of this cell.

It is further known that by using high-throughput gene expression measurements (microarray data) these gene expression rates can be measured. The microarray data in its turn describes snapshots of the gene expression pattern.

Many illnesses and malfunctions of the body are attributable to disturbances in the regulatory genetic network which is reflected by greatly changed gene expression behavior (gene expression rates) or a changed gene expression pattern of a cell.

An understanding of the regulatory genetic network thus represents an important step on the path to a characterization of the understanding of genetic mechanisms as well as consequently of identification of what are known as dominant or malfunction-initiating genes underlying the illnesses or malfunctions.

In cancer research for example suppressing genes can play a key role in the identification of growths and tumors, the knowledge of new potential oncogenes and their interactions with other genes can be a contribution to discovering the basic principles (of cancers) which determine how normal cells change into malignant cancer cells.

Furthermore a quantitative understanding of the regulatory genetic network of a cell is necessary for developing improved medicaments and therapies for fighting genetic diseases.

Thus a number of medicaments act as agonists or antagonists of specific target proteins, i.e. they strengthen or weaken the function of a protein with corresponding effect on the regulatory genetic network with the aim of bringing this back into a normal function mode.

A description of a regulatory genetic network of a cell using a statistical method, a causal network is known from DE 10159262.0.

A causal network, a Bayesian network, is known from Jensen, An Introduction to Bayesian Networks, UCL Press, London, 1996.

Bayesian Networks

A Bayesian network B is a specific type of presentation of a common multivariate probability density function (WDF) of a set of variables X by a graphical model which consists of two parts.

It is defined by a directed acyclic graph, DAG) G—of the first component, in which each node i=1, . . . , n corresponds to a random variable Xᵢ.

The connectors between the nodes represent statistical dependencies and can be interpreted as causal relationships between them. The second component of the Bayesian network is the set of conditional WDFs P(Xᵢ|Paᵢ,θ,G), which are parameterized by a vector θ.

These conditional WDFs specify the type of dependencies of the individual variables i of the set of its parents _{Pa}i. Thus the common WDF can be broken down into the product form

$$P \text{(} {X}_{1} , {X}_{2} , … {X}_{n} = {\prod }_{i = 1}^{n} P ( {X}_{i} \vert {Pa}_{i} , θ , G )$$

The DAG of a Bayesian network uniquely describes the conditional dependency and independency relationships between a set of variables, but by contrast a given statistical structure of the WDF does not result in any unique DAG.

Instead it can be shown that two DAGs describe one and the same WDF, if and only if they feature the same set of connectors and the same set of “colliders”, with a collider being a constellation in which at least two directed connectors lead to the same node.

### SUMMARY OF THE INVENTION

An object of the invention is to specify a method which allows an analysis of a regulatory genetic network of a cell, for example represented by at least one gene expression pattern of the cell.

A further object of the invention is to specify a method which enables a defective gene to be identified, for example a cancer or tumor gene, in the regulatory genetic network of a cell.

Further the invention is designed to allow a simulation and/or an analysis of an effect of a medicament on the regulatory genetic network of a cell.

In the basic method for analysis of a regulatory genetic network of a cell a causal network is used,
the causal network describing the regulatory genetic network of the cell such that nodes of the causal network represent genes of the regulatory genetic network and connectors of the causal network represent regulatory interactions between the genes of the regulatory genetic network

In the analysis method a gene expression rate is now specified for a selected gene of the regulatory genetic network. Using the causal network a resulting gene expression pattern is generated for the predetermined gene expression rate for the regulatory genetic network. The resulting gene expression pattern generated is subsequently compared with a predetermined gene expression pattern of the regulatory genetic network.

A probabilistic semantic of a causal network, such as of a Bayesian network, is very well suited to analysis of gene expression rates, given for example in the form of microarray data, since it is adapted to the stochastic nature both of biological processes and also to experiments susceptible to noise.

Furthermore, viewed in illustrative terms, an effect of an expression state of specific genes on a global gene expression pattern (inverse modeling) is estimated, in that a resulting gene expression pattern is analyzed.

The developments described below relate to both the method and to the configuration.

The invention and the developments described below can be implemented both in software and also in hardware, for example by using a specific electrical circuit.

With a further development the selected gene is selected using the causal network by a dependency analysis.

The gene expression rate of the selected gene can also be predetermined such that the predetermined gene expression rate of the selected gene reflects an assumption of a gene defect.

A Bayesian network can be used as the causal network.

The causal network can also be of a type DAG (Directed Acylic Graph).

Furthermore the generated resulting and/or the predetermined gene expression pattern can represent discrete gene states, with the represented discrete gene states being able to be a an overexpressed, a normal or an underexpressed gene state.

In a further development the generated resulting gene expression pattern can be compared with the predetermined gene expression pattern using a static method and/or of a statistical code, especially a measure of distance.

There can also be provision for the causal network to be trained using gene expression patterns, with the nodes and the connectors of the causal network being adapted.

Furthermore it is expedient for the gene expression patterns, especially the predetermined gene expression pattern and/or the gene expression patterns for training, to be determined using a DNA microarray technique.

In one embodiment the predetermined gene expression pattern and/or the gene expression pattern for training is a gene expression pattern of a genetic regulatory network of a diseased cell.

Here for example the diseased cell can be a cancer cell, especially a oncocell with ALL (Acute Lymphoblastic Leukemia).

Furthermore the diseased cell can feature an oncogene, especially an ALL oncogene.

Also for a plurality of selected genes of the regulatory genetic network one gene expression can be predetermined in each case, a plurality of resulting gene expression patterns generated and/or a plurality of comparisons undertaken.

In a further development the generation of the plurality of resulting gene expression patterns is performed iteratively.

Furthermore the inventive procedure or development is particularly suitable for identifying a dominant gene and/or a degenerated/mutated/diseased gene/oncogene/tumor-suppressor gene.

It is also suitable for identifying a tumor cell, for example in connection with cancer detection.

Further the inventive method is especially suited to analyzing the causes of an abnormal gene expression pattern/ gene expression rate.

It can also be used for a simulation and/or analysis of the effects of a medicament.

### BRIEF DESCRIPTION OF THE DRAWINGS

These and other objects and advantages of the present invention will become more apparent and more readily appreciated from the following description of an exemplary embodiment of the invention, taken in conjunction with the accompanying drawings of which:

FIG. 1 is a flowchart of a procedure for investigating genetically-related causes of illness through Bayesian inverse modelling using a cancer as an example;

FIG. 2 is a procedural listing for an algorithm for creating a data set of N samples in accordance with an exemplary embodiment;

FIG. 3 is a procedural listing for a procedure for creating data sets, which reflect an effect of different observations in accordance with an exemplary embodiment;

FIGS. 4a and 4b are graphs which show that data obtained by sampling show subtype characteristic expression patterns as also in an original data set;

FIG. 5 is a graph which shows graphically a probability of each subtype under a condition which is overexpressed on a gene, for all 271 genes;

FIG. 6 is a graph structure of a causal network, which represents a regulatory genetic network.

### DETAILED DESCRIPTION OF THE PREFERRED EMBODIMENT

Reference will now be made in detail to the preferred embodiments of the present invention, examples of which are illustrated in the accompanying drawings, wherein like reference numerals refer to like elements throughout.

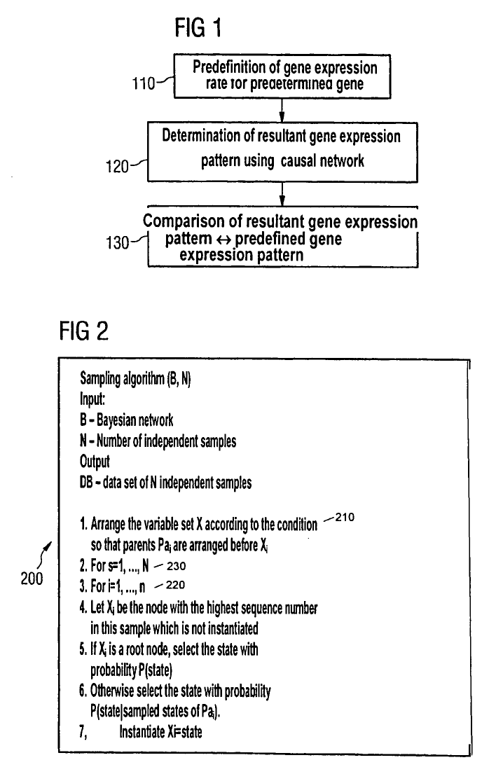

### Exemplary Embodiment Investigation of Genetically-Related Causes of Diseases Using Bayesian Inverse Modelling Using a Cancer as an Example (Espec. FIG. 1)

Overview of the Bayesian Inverse Modelling (BIM) Procedure

In many areas of empirical research the desire is to reach conclusions from the observation of trial results about the underlying principle and its causes—the relationship between “cause” and “effect”.

For example in cancer research the underlying principle is studded which causes a normal cell to transform it into a malignant, rapidly growing cancer cell.

The effect of the various types of cancer is known, e.g. the general appearance of a cancer cell compared to a normal cell, measured with the aid of microarray chips.

By contrast the cause of its origination is largely unknown.

On the basis of the understanding that cancer is a genetic illness and that it is attributable to a deviation in the behavior of cells, the research is concentrating on discovering the genetic principles which are responsible for the development of the cancer.

An important task in this environment is to identify genes which can play a role in tumor genesis, such as for example growth and tumor-suppressing genes.

A procedure is described below with which it is possible to identify genes which are a potential cause of tumor genesis.

One element of the procedure is a statistical method, in this case a Bayesian network (see Jensen, above and subsequent associated embodiments for more details), which is learnt (see DE 10159262.0) from a microarray data set as described in Stetter (see “Structural learning” below) (cf. FIG. 1).

In this case it is assumed that the set of the measured gene expression vectors X belong to a basic totality with a highly-dimensional multivariate probability density function which is modelled with the aid of Bayesian network with adaptive network structure.

The relationships between the variables, namely the conditional dependences and independences, are represented by a Directed Acyclic Graph (DAG) G.

The probabilistic semantic of the Bayesian network is very well suited to the analysis of microarray data since it is adapted to the stochastic nature both of the biological processes and also of the experiments susceptible to noise.

In the procedure described below the learnt Bayesian network will be used as a generative model for taking samples of artificial microarray data sets which supplies the learned conditional probability density distributions (cf. FIG. 1, step 110-130).

Furthermore the effect of the expression state of specific genes on the global gene expression pattern (inverse modelling) is estimated, in that a resulting data set is analyzed (cf. FIG. 1, step 110-130).

In the procedure described below each gene is also assigned its probability, with which it is the cause of these cell states.

To this end these data sets are compared with data obtained from microarray investigations of various known cell states (cf. FIG. 1, step 130).

Seen in general terms, the procedure does not concentrate explicitly on the structures of the network, but rather on the probability distribution which is derived from the learnt Bayesian network.

Finally the procedure is applied to microarray data of different subtypes of pediatric acute lymphoblastic leukemia (ALL) of Yeoh et al., “Classification, Subtype Discovery, and Prediction of Outcome in Pediatric Acute Lymphoblastic Leukemia by Gene Expression Profile”, Cancer Cell, 2002, pp. 133-143.

The comparison of the artificial data with expression patterns of specific cancer subtypes enables a measure of probability of the illness-causing behavior of each gene (cf. FIG. 1, step 130) to be obtained.

Results of the applied procedure show that, in connection with Bayesian Inverse Modelling (BIM) this allows the effect of pathogenetically modified expression levels on the global gene expression pattern to be predicted, in which case already known oncogenes as well as potential new ones are found.

Bayesian Networks

The basic principles of Bayesian networks as described in Jensen have already been described above.

In the case of the modelling of a regulatory genetic network by a Bayesian network genes or their corresponding proteins are symbolized by nodes.

Regulation mechanisms are described by connectors between two nodes, which can be interpreted in a causal manner.

The quality of the regulation is encoded in the conditional probability distribution of the gene involved for given regulators of the same.

Structural Learning

The process of structural learning can be described as follows:

Let D={d¹, d², . . . , d^{N} be a data set of N independent observation, with each data point being an n-dimensional vector with components d¹={d^{I}, d₂, . . . , d¹_{N}). For a given D the structure G of the Bayesian network is to be found which best corresponds to D, i.e. which maximizes the Bayes-Score,

$$S \text{(} Q \vert D ) = \frac{P ( D \vert G ) P ( G )}{P ( D )}$$

with P(D|G) the being the peripheral probability, P(G) the apriori probability of the structures and P(D) the evidence.

Since both the apriori probability and also the evidence are unknown, the problem is reduced to determining the structures with the best peripheral probability corresponding to the data (Heckerman et al., “Learning Bayesian networks: The combination of knowledge and statistical data”, Machine Learning, vol. 20, 1995, pp. 197-243).

If the data set D consists of N microarray experiments, e.g. of cell samples of different patients, each data vector {d¹₁, d¹₂, . . . , d¹ₙ} represents the expression profile of n genes in a microarray experiment.

A Bayesian network learnt from such data encodes the probability distribution of n genes, which were obtained from these N microarray experiments.

Bayesian Inverse Modelling (BIM)

Generative Model

A learnt (see notes above about “structural learning”) Bayesian network B represents a density estimation function which reflects the probability distribution of the data set D, on the basis of which it was learnt, with the aid of the set of conditional WDFs.

This means that it can be used as a generative model for creating a data set D_{B} which reflects the density distribution obtained from D.

FIG. 2 shows an algorithm 200 for creating a data set of N samples from B.

The first step 210 of the algorithm 200 consists of arranging all variables such that the parents (parent nodes) Paᵢ are instantiated before Xᵢ.

Subsequently the variables corresponding to the arrangement are selected and instantiated with a value 220.

The value of each variable is selected with the probability P(state|Paᵢ). This step is repeated 230, until N samples are created.

Probabilistic Interference

A significant problem in Bayesian networks is the evidence propagation, meaning the determination of the aposteriori distribution P(X_{q}|E) of a request variable X_{q}, if a certain evidence E has been observed in the Bayesian network.

As a result of the definition of a conditional probability, the aposteriori probability is

$$P \text{(} {X}_{q} \vert E ) = \frac{P ( {X}_{q} , E )}{P ( E )} = \frac{\sum x \backslash ( {x}_{q} , {x}_{E} ) P ( X )}{\sum X \backslash {X}_{E} P ( X )}$$

with X_{E} designating the quantity of the observed variables.

To overcome the time complexity, the different methods of exact interference calculation use the general principle of dynamic programming.

As part of this exemplary embodiment a simple interference algorithm, of “bucket elimination”, as described in Dechter, R., “Bucket Elimination: A unifying framework for probabilistic inference”, Uncertainty in Artificial Intelligence, UAI 196, pp. 211-219, is used.

The basic idea with this interference algorithm consists of eliminating variables one after the other in accordance with an order of elimination p by summation.

In this way P(X_{q}|E) can be efficiently calculated within a perceivable time.

Interventional Modelling by Setting the Evidence

With the interventional modelling approach the effect of specific observation on the behavior of the Bayesian network using a combination of probabilistic interference and data sampling is estimated.

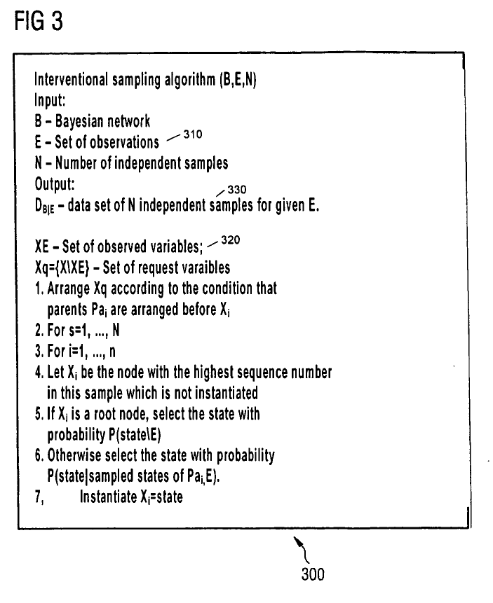

In accordance with FIG. 3 the Bayesian network can be viewed as a kind of black box 300, with the input being given by a set of observations E 310 and the corresponding list of observed variables X_{E} 320.

The output, which is given by the data set D_{B|E} 330 is created using the method previously explained in association with FIG. 2.

In addition the empirical evidence is to be taken into account.

Consequently each state of Xᵢ is selected with probability P(state|Paᵢ,E), which is calculated by probabilistic interference.

With the procedure described in accordance with FIG. 3 different data sets can now be created which reflect the effect of the different observations.

If, as described below, biological effects are analyzed, this means that through this method of operation in accordance with FIG. 3 artificial microarray data can be created which reflects the probability distribution of a certain data set if specific observations are given.

If the artificially created data from a known origin is compared for example with a cancer-specific set of measurement data, those genes can be determined which, when they are fixed at a certain expression level, will influence the model so that these two microarray data sets, the artificial and the known, exhibit the same characteristics.

Statistical Comparison of Data Sets

In order to estimate the quality of the influence of the evidence I on the behavior of the Bayesian network I, the created data set D_{B|E} is compared with a set of data sets I of known states S.

It is assumed that D describes the effect of different types of cancer. In accordance with the embodiment the behavior of evidence E relating to a specific type of cancer S can now be described.

By using a measure of distance the change a of the correlation between D_{B|E and Ds as a result of E can be estimated:}

$$a ( E ) = \frac{ⅆ ( {D}_{B \vert E} , {D}_{S} )}{ⅆ ( {D}_{B} , {D}_{S} )}$$

with the distance between the two data sets having been standardized with the aid of the distance between D_{B}, which was taken from B without evidence, and Ds.

As a result, in accordance with the embodiment, the influence of an observed evidence is measurable, e.g. the expression state of a specific gene on a behavior of the model characteristic for cancer.

Secondly the probability can be calculated of B creating a data set D_{B|E} which is equal to Ds for a given E.

For this purpose an estimate is made of how many samples d^{I} of D_{B|E} lie closest to Ds in that the distance between each sample and each data set is calculated by D.

The aposteriori probability P(S|E) of the occurence of the cancer type S for given evidence E is thus obtained:

$$P \text{(} S \vert E ) = \frac{{N}_{ES}}{N} \qquad ( 5 )$$

with Nₑₛ being a number of samples of DB|E, which is statistically closest to the data set DS, and with N being the total number of samples of D_{B|E}.

As already pointed out above, empirical research deals with the relationship between cause and effect, in that it draws conclusions about the underlying cause from experimental observation.

With the Bayesian Inverse Modelling approach in accordance with the exemplary embodiment an underlying cause is estimated by first creating an effect which stems from a known observation.

After this inverse step this effect is compared with effects which are well-defined but for which the cause is unknown.

The potential cause of the best-match effect is then given by the observation which gives rise to the created effect.

The ALL Microarray Data Set of Yeoh et al.

The data which is used for the analysis in accordance with the exemplary embodiment consists of 327 samples of various subtypes of pediatric acute lymphoblastic leukemia (ALL).

The data set was assembled by Yeoh and his colleagues at the St. Jude Children's Research Hospital.

ALL is a heterogeneous illness which includes different subtypes, including both T-cell type leukemia and B-cell type leukemia, which differ as regards their reaction to a medical treatment.

Apart from T-ALL, of which the cause is not clearly known, each B-cell subtype can be traced back to a specific genetic modification, e.g. to genetic translocations t(9;22) [BCR-ABL], t(1;19) [E2A-PBX1], t(12;21) [TEL-AML1], t(4;11) [MLL] or to a hyperdiploid karyotype [>50 chromosomes].

No wonder then that the gene expression patterns of the different subtypes differ very markedly from one another.

Furthermore microarray data exhibits one more clear expression profile which points to the existence of a further ALL subtype in addition to the 6 known.

It should be pointed out that Yeoh et al. are working on a robust classification for classifying the subtypes using a support vector machine with a set of 271 discriminating genes.

Results

Learnt Structure

For analysis in accordance with the exemplary embodiment the reduced data set of 271 genes and 327 samples of different ALL subtypes, as described above with respect to the work by Yeoh et al., is used.

To perform the learning process of a multivariate model the data set in the values has been divided up into the discrete value “under-expressed”, “expressed normally” and “over-expressed”.

The learnt structure shows scale-free characteristic values, a feature which is typical of biological networks, such as for metabolic networks or signaling networks.

Such networks are characterized by a power distribution of the ranges of a node which is defined as the number of connections to other nodes.

These nodes have a strong influence on the dynamics and robustness of scale-free networks, and of many of these strongly connected genes in our model it is actually known that they play a role in the ocogenesis or in the critical processes associated with the development of cancer, e.g. DNA repair.

First a data set of 300 samples is now created from the model in order to estimate the statistics which are defined by the set of the conditional probabilities.

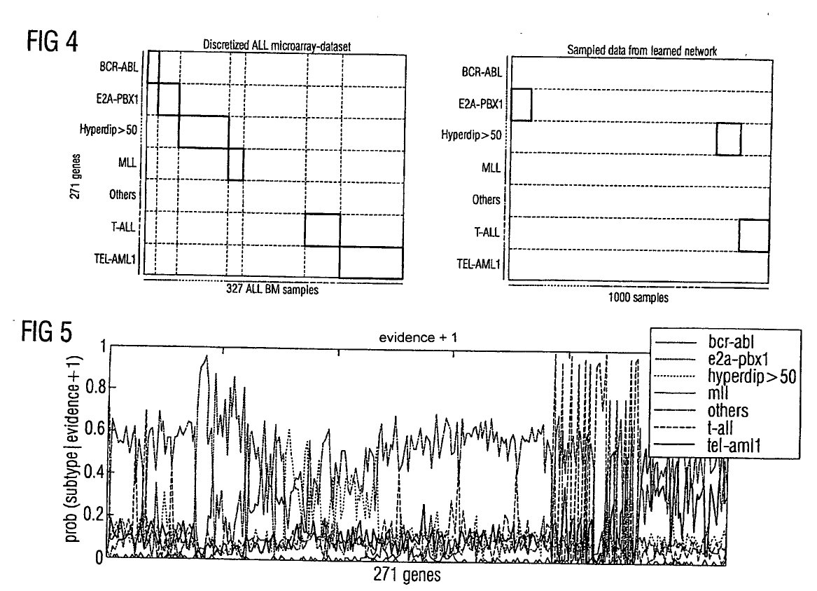

FIGS. 4a and 4b show that data obtained by taking samples (FIG. 4b) shows subtype characteristic expression patterns, as is also the case in the original data set (FIG. 4a).

The patterns of a number of subtypes such as E2A-PBX1 or T-ALL, are reproduced very well whereas others are generated less well, e.g. the pattern of the subtype MLL, or are missed completely such as for example BCR-ABL.

Modelling of Leukaemia Subtypes by Intervention

The learnt Bayesian network is the basic starting point for the exemplary embodiment for the approach adopted of using inverse modelling to find those genes which, when fixed at a specific expression level, influence the model such that the generated artificial microarray data set exhibits specific characteristics.

As described above, the probability P(C|E) of creation of specific cancer subtype C is estimated if a certain observation E is given, in this case the expression state of a specific gene P(C|Genᵢ=state).

By contrast with Yeoh, not only the presence of a specific cancer subtype is predicted, but genetic mechanisms which lead to its creation.

A high probability indicates that the fixed gene is a potential cause for the subtype-specific expression behavior of the gene in question, which in its turn can be the underlying cause of a specific cancerous appearance.

7 reference data sets are used for the comparison, with each of these having been obtained in conjunction with a specific ALL subtype.

FIG. 4a shows that the original microarray data set is clearly subdivided into 7 clusters (accumulations of points) with different sample extents.

Each of these clusters represents the expression pattern of 271 genes if a specific subtype of leukaemia is given, and has been used to to measure the influence of an evidence for the occurrence of these different ALL subtypes.

In a first step each gene is fixed for any one of its expression values, with all these conditions being used to to generate a data set of 300 samples (FIG. 4b).

Subsequently all this data is compared with the 7 reference data sets, as explained previously.

In FIG. 5 the probability of each subtype, under the condition that a gene is overexpressed, is shown on a graph for 271 genes.

FIG. 5 shows that a small number of genes exist which are very likely to trigger a specific ALL subtype if they are strongly active.

To verify these results the molecular function of specific genes and their role in biological processes, especially as regards pathogenesis, is examined in more detail below.

Biological Insights

These are obtained by examining in greater detail the genes which are very probably the cause of a specific subtype as well as significant structure patterns in the learnt network, i.e. dominant genes and their environment.

The learnt Bayesian network (model) results from the microarray data set of different leukaemia subtypes and reflects transcriptional relationships between genes which occur in these malignant cancer cells.

Thus genes which trigger a specific subtype are either potential oncogenes or are regulated by such genes.

The first gene to be analyzed in more detail is the gene PBX1.

If it is overexpressed the learnt Bayesan network creates a data set with 0.96 probability which is characteristic of the subtype E2A-PBX1 of the ALL off B-cell type (see FIG. 5).

This makes the obvious assumption that a causal relationship between the “overexpression” of this gene and the occurrence of the ALL subtypes E2A-PBX1 is present.

And in actual fact PBX1 s known as a proto ocogene which causes normal blood cells to mutate into malignant ALL cancer cells.

As a result of the chromosome translocation t(1;19) PBX1 merges with the gene E2A and transform into a potent ocogene which causes the leukemia subtype E2A-PBX1.

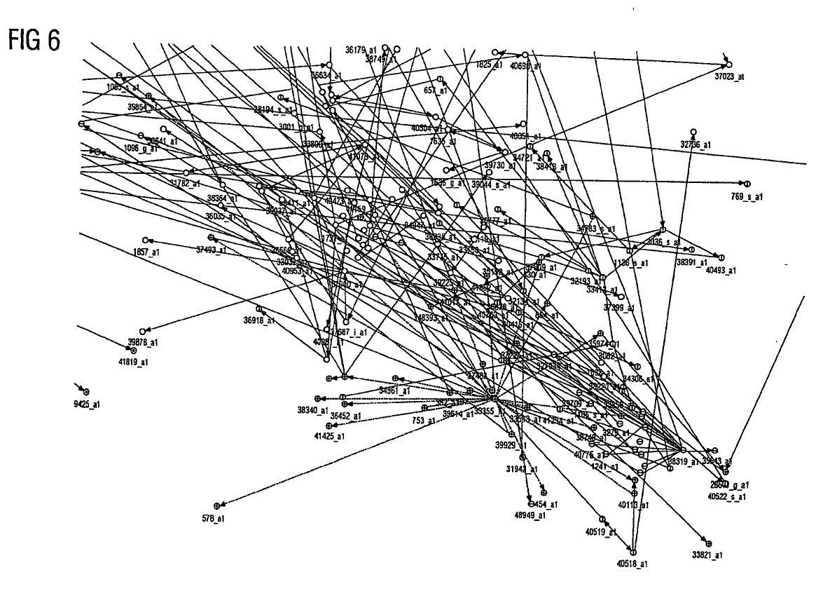

Since the graph structure of the model (FIG. 6) can further be interpreted in a causal manner it provides information about the interaction between potential oncogenes and other genes which in its turn can be interpreted as an oncogene regulation.

|the structure of the network (FIG. 6) is considered, PBX1 represents a dominant gene in that it influences many other genes but is only regulated by one or a few other genes.

In addition, as a result of the conditional probability distribution, the model identifies PBX1 as a transcription activator.

This can also be explained by known biological facts, since PBX1 activates genes which are normally not expressed or are expressed at a low level.

Patients with a hyperdiploidy of >50 chromosomes have clones of 51-68 chromosomes. Although high hyperdiploid clones are seldom identical, they tend to exhibit a pattern of the chromosome increase with additional copies of the chromosomes 4, 6, 10, 14, 18 and 21.

Trisomy and Polysomy 21 are non-random anomalies which are frequently to be observed with ALL Their occurrence, even if it is not specific, as well as the increased occurrence of acute leukaemia or in subjects with constitutional Trisomy 21 make it reasonable to assume that the chromosome 21 has a particular role to play in leukemogenesis.

Another disease, Down's Syndrome, is caused by Trisomy 21 and shows an increased occurence of leukemia such as ALL.

As a result the method described makes it possible in this case, in accordance with the exemplary embodiment, to identify genes which to a large extent indicate the hyperdiploid ALL subtype, of which however it is also known that they play a significant role in the occurrence of Down's Syndrome.

The gene SOD1 is located at chromosome 21 and produces an enzyme which converts superoxide-free radicals into hydrogen peroxide. The increased expression at Trisomy 21, which is also to be observed for the microarray samples of patients with hyperdiploid karyotype, can give rise to the brain damage which is to be seen with Down's Syndrome.

The frequency of the occurence of the hyperdiploid ALL also increases in the case in which the gene PSMD10 is overexpressed.

PSMD10 is a regulatory cluster unit of the proteasome 26S for which it has been shown that is operates as a natural mechanism for the breakdown of protein by regulating the protein metabolism in eukaryotic cells

This is of significance for cancers in humans since the cell cycle, the growth of the tumor and the survival are determined by a great vraiety of intracellular proteins which are regulated by the ubiquitin-dependent proteasome breakdown path which is influenced by PSMD10.

In more recent scientific work it has been verified that this breakdown path is often the object of a deregulation associated with cancer and can be subject to such processes as oncogene transformation, tumor progression, bypassing of the immune system and resistance to medicaments.

Abstract of the Exemplary Embodiment

The exemplary embodiment described presents a new method by which it is possible to identify genes which are a potential cause of tumorgenesis, by analyzing the relationships between microarray data of leukemia subtypes and a data set, which is the result of taking samples from a learnt Bayesian network.

This method of operation is based on the modelling of a regulator genetic network through a Bayesian network, with genes or their corresponding proteins being symbolized by the nodes of the Bayesian network.

Regulation mechanisms are described by connectors between two nodes, which can be interpreted in a causal manner.

The quality of the regulation is encoded in the conditional probability dsitribution of the gene involved for given regulators of the same.

The understanding of the regulatory genetic network represents an important step along the road to characterizing the genetic mechanisms underlying complex diseases.

In cancer research, were the identification of genes which suppress growths and tumors plays a key role, the knowledge of new potential oncogenes and their interactions with other molecules is an important contribution to discovering the basic principles which determine why normal cells mutate into malignant cancer cells.

With the procedure described in accordance with the exemplary embodiment, especially with Bayesian Inverse Modelling, it is possible to discover genes with such an oncogene characteristic simply through a statistical analysis of gene expression patterns, which have been measured with the aid of DNA microarrays.

The underlying theoretical probability model which has been used, is a Bayesian network, which encodes the multivariate probability distribution of a set of variables by a set of conditional probability distributions.

The statistical dependencies are encoded in a graph structure. In the learning method Bayesian statistics are used to determine the network structure and the corresponding model parameters which best describe the probability distribution contained in the data.

The invention has been described in detail with particular reference to preferred embodiments thereof and examples, but it will be understood that variations and modifications can be effected within the spirit and scope of the invention covered by the claims which may include the phrase “at least one of A, B and C” as an alternative expression that means one or more of A, B and C may be used, contrary to the holding in Superguide v. DIRECTV, 69 USPQ2d 1865 (Fed. Cir. 2004).

1-24. (canceled)

25. A method for analysis of a regulatory genetic network of a cell using a causal network describing a regulatory genetic network of cells such that nodes of the causal network represent genes of the regulatory genetic network and connectors of the causal networks represent regulatory interactions between the genes of the regulatory genetic network, said method comprising:
providing a predetermined gene expression rate for a selected gene of the regulatory genetic network;
generating a resulting gene expression pattern for the regulatory genetic network using the causal network for the predetermined gene expression rate; and
comparing the resulting gene expression pattern with a predetermined gene expression pattern of the regulatory genetic network.

26. A method in accordance with claim 25, further comprising selecting the selected gene by dependency analysis using the causal network.

27. A method in accordance with claim 26, wherein the predetermined gene expression rate of the selected gene reflects an assumption of a gene defect.

28. A method in accordance with claim 27, wherein the causal network is a Bayesian network.

29. A method in accordance with claim 28, wherein the causal network is a directed acylic graph type.

30. A method in accordance with claim 29, wherein at least one of the resulting gene expression pattern and the predetermined gene expression pattern represents discrete gene states.

31. A method in accordance with claim 30, wherein the discrete gene states include an overexpressed gene state, a normally expressed gene state and an underexpressed gene state.

32. A method in accordance with claim 31, wherein said comparing of the resulting gene expression pattern to the predetermined gene expression pattern uses at least one of a static method and a statistical code as a measure of distance.

33. A method in accordance with claim 32, further comprising training the causal network using training gene expression patterns to adapt the nodes and the connectors of the causal network.

34. A method in accordance with claim 33, further comprising determining at least one of the predetermined gene expression pattern and the training gene expression patterns using a DNA microarray technique.

35. A method in accordance with claim 34, wherein at least one of the predetermined gene expression pattern and the training gene expression patterns are for a diseased cell.

36. A method in accordance with claim 35, wherein the diseased cell is an oncocell.

37. A method in accordance with claim 36, wherein the diseased cell features an Acute Lymphoblastic Leukemia oncogene.

38. A method in accordance with claim 25, further comprising repeating said determining, said generating and said comparing to determine a plurality of predetermined gene expression rates for selected genes of the regulatory genetic network and to generate and compare the resulting gene expression pattern for each of the predetermined gene expression rates with a corresponding predetermined gene expression pattern.

39. A method in accordance with claim 38, wherein said repeating of the generation the resulting gene expression patterns is performed iteratively.

40. A method in accordance with claim 39, further comprising identifying a dominant gene based on said comparing repeatedly performed.

41. A method in accordance with claim 39, further comprising identifying at least one of a degenerated gene, a mutated gene, a diseased gene, an oncogene, and a tumor-suppressor gene based on said comparing repeatedly performed.

42. A method in accordance with claim 39, further comprising identifying a tumor cell based on said comparing repeatedly performed.

43. A method in accordance with claim 39, further comprising detecting cancer based on said comparing repeatedly performed.

44. A method in accordance with claim 39, further comprising analyzing a cause of an abnormal gene expression pattern/gene expression rate based on said comparing repeatedly performed.

45. A method in accordance with claim 39, further comprising simulating an effect of a medicament based on said comparing repeatedly performed.

46. A method in accordance with claim 39, further comprising analyzing an effect of a medicament based on said comparing repeatedly performed.

47. At least one computer-readable medium storing a program which when executed on a computer causes the computer to perform a method comprising:
providing a predetermined gene expression rate for a selected gene of the regulatory genetic network;
generating a resulting gene expression pattern for the regulatory genetic network using the causal network for the predetermined gene expression rate; and
comparing the generated resulting gene expression pattern with a predetermined gene expression pattern of the regulatory genetic network.
